(19) United States
(12) Patent Application Publication
(10) Pub. No.: US 2004/0014761 A1
(43) Pub. Date: Jan. 22, 2004

(54) Treatment of female sexual dysfunction with phosphodiesterase inhibitors

(76) Inventors: Virgil A. Place, Kawaihae, HI (US); Leland F. Wilson, Menlo Park, CA (US); Paul C. Doherty JR., Cupertino, CA (US); Mark S. Hanamoto, Belmont, CA (US); Alfred P. Spivack, Menlo Park, CA (US); Neil Gesundheit, Los Altos, CA (US); Sean R. Bennett, Denver, CO (US); Jane Doherty, Cupertino, CA (US)

Correspondence Address: REED & EBERLE LLP, 800 MENLO AVENUE, SUITE 210, MENLO PARK, CA, 94025, US

(21) Appl. No.: 10/279,039

(22) Filed: Oct. 22, 2002

Related U.S. Application Data
(63) Continuation-in-part of application No. 09/499,959, filed on Feb. 8, 2000, now Pat. No. 6,469,016.
Application No. 09/499,959 is a division of application No. 09/181,316, filed on Oct. 27, 1998, now abandoned.
Application No. 09/181,316 is a continuation-in-part of application No. 08/959,064, filed on Oct. 28, 1997, now Pat. No. 5,877,216.
Application No. 09/181,316 is a continuation-in-part of application No. 08/959,057, filed on Oct. 28, 1997, now abandoned.

(51) Int. Cl. A61K 31/519; A61K 31/4965; A61K 31/50; A61K 31/4709

(52) U.S. Cl. 514/247; 514/259.41; 514/264.1; 514/314; 514/389; 514/255.06

(57) ABSTRACT

A topical pharmaceutical composition is provided for the treatment of female sexual dysfunction, wherein the composition is formulated so as to contain a therapeutically effective amount of a phosphodiesterase inhibitor and a pharmaceutically acceptable carrier for topical administration. The phosphodiesterase inhibitor is generally selected from Type III, Type IV, Type V, and nonspecific phosphodiesterase inhibitors.

### CROSS-REFERENCE TO RELATED APPLICATIONS

This application is a continuation-in-part of U.S. Ser. No. 09/499,959, issuing Oct. 22, 2002 as U.S. Pat. No. 6,469,016, which is a divisional of U.S. Ser. No. 09/181,316, filed Oct. 27, 1998, abandoned, which was a continuation-in-part both of U.S. patent application Ser. No. 08/959,064, filed Oct. 28, 1997, which issued on Mar. 2, 1999 as U.S. Pat. No. 5,877,216, and of U.S. patent application Ser. No. 08/959,057, also filed Oct. 28, 1997, abandoned. The disclosures of each of the aforementioned are hereby incorporated by reference.

### TECHNICAL FIELD

This invention relates generally to pharmaceutical formulations and methods for treating female sexual dysfunction, and more particularly relates to vaginal and/or vulvar administration of a formulation containing a phosphodiesterase inhibitor in such treatment.

### BACKGROUND

Sexual response in women is generally classified into four stages: excitement, plateau, orgasm, and resolution. Masters and Johnson, Human Sexual Response (Boston, Mass.: Little, Brown & Co., 1966). With sexual arousal and excitement, vasocongestion and muscular tension increase progressively, primarily in the genitals, and is manifested by increased blood flow, elevated luminal oxygen tension, and vaginal surface lubrication as a result of plasma transudation that saturates the fluid reabsorptive capacity of the vaginal epithelium. Sexual excitement is initiated by any of a number of psychogenic or somatogenic stimuli and must be reinforced to result in orgasm. With continued stimulation, excitement progresses in intensity into a plateau stage, from which the individual can shift into orgasm. The orgasmic stage is characterized by a rapid release from vasocongestion and muscular tension.

During the various stages of sexual response, characteristic genital and extragenital responses occur. Estrogens magnify the sexual responses; however, sexual responses may also occur in estrogen-deficient individuals. Sexual dysfunction may be due to organic or functional disturbances. For example, a variety of diseases affecting neurologic function, including diabetes mellitus and multiple sclerosis, may interrupt sexual arousal. More commonly, local pelvic disorders, such as endometriosis and vaginitis, both of which cause dyspareunia (difficult or painful coitus) may also affect a woman's sexual response. In addition, estrogen deficiency, causing vaginal atrophy and dyspareunia, is a common cause of sexual dysfunction. For a discussion of other causes of female sexual dysfunction, see, e.g., Kaplan, The Evaluation of Sexual Disorders: Psychological and Medical Aspects (New York: Brunner-Mazel, 1983), and Kolodny et al., Textbook of Sexual Medicine (Boston, Mass.: Little, Brown & Co., 1979).

Excitement stage dysfunction generally involves touch sensation impairment, loss of clitoral sensation, vaginal dryness and urinary incontinence. Such excitement phase dysfunction generally results in dyspareunia. Dyspareunia is thought to affect approximately 40% of women, due in large part to inadequate lubrication. It has been estimated that over 40 million women will suffer dyspareunia at some time in their lives. On the order of twenty-five million will experience dyspareunia in the peri- and postmenopausal period (see Kelly, S. (1992) Clinical Practice and Sexuality 8(8):2 and Sato et al. (1992) Clinical Practices in Sexuality 8(5): 1). Contemporary symptomatic treatments generally involve the use of physiologically safe lubricants such as egg white, K-Y surgical lubrication jelly (hydroxyethyl-cellulose), Astroglide®, and Replens®. See, for example, Semmens (1974) Medical Aspects of Human Sexuality 8:85-86, and Frishmen et al. (1992) Fertility and Sterility 58(3):630. When symptomatic treatment fails, pharmacological treatment may be indicated.

Estrogen therapy is commonly used in the pharmacological treatment of sexual dysfunction in women. Estrogen-based therapies are generally used to increase mucous production, provide vasodilatory effects, or to increase the general health of the vagina. Nadelson et al., eds., Treatment Interventions in Human Sexuality (New York: Plenum Press, 1983). In such treatments, estrogen is administered orally, parenterally (e.g., by injection), or topically. With oral administration, the estrogen concentration encountered by the liver is generally four- to five-fold greater than estrogen levels in peripheral blood (the “first pass effect”). This effect may lead to an undesirable increase in the production of certain coagulation factors and renin substrates by the liver. Parenterally administered estrogen avoids the first pass effect in the liver. However, all estrogen-based therapies are known to increase the risk of endometrial hyperplasia endometrial cancer and breast cancer in treated individuals.

Because of the increased risk of endometrial hyperplasia and endometrial cancer encountered with unopposed estrogen therapies, estrogen/progestogen combinations have been employed. However, progestogens are known to have some androgenic activity. Further, common side effects from such therapies include uterine bleeding and the continuation of menstrual periods. Accordingly, there remains a need in the art to provide safer and more ways of treating female sexual dysfunction.

The present invention is directed to the aforementioned need in the art, and provides a new, highly effective method of treating sexual dysfunction in women.

### SUMMARY OF THE INVENTION

Accordingly, it is a primary object of the invention to address the aforementioned need in the art by providing a novel treatment for female sexual dysfunction involving topical administration of a pharmaceutical composition containing a phosphodiesterase inhibitor. It will be appreciated by those working in the field of sexual pharmacotherapy that the present method of “treating sexual dysfunction” also encompasses prevention of sexual dysfunction, e.g., enhancement of sexual desire and responsiveness in a woman who is not suffering from clinically identifiable “sexual dysfunction” per se.

In one embodiment, then, a pharmaceutical composition is provided for treatment of female sexual dysfunction, the formulation comprising (a) a therapeutically effective amount of a phosphodiesterase inhibitor, such as a Type III, Type IV, or Type V phosphodiesterase inhibitor; and (b) a pharmaceutically acceptable topical carrier. The topical carrier may be one that provides release of the phosphodiesterase inhibitor from the composition upon contact with the body surface (i.e., with the mucosal surface of an individual's vulvar region and/or vagina). Such an immediate release formulation enables “as-needed” administration, i.e., administration at some point just prior to sexual activity, typically in the range of about 1 minute to about 60 minutes, more typically in the range of about 5 minutes to about 60 minutes, prior to sexual activity. The carrier may also provide for sustained release, in which case release of the active agent occurs throughout an extended time period. The therapeutically effective topical dosage of the phosphodiesterase inhibitor will, of course, vary with the particular inhibitor, dosage form, and other factors, but will generally be in the range of about 5 μg to about 125 mg per dose, more typically in the range of about 50 μg to about 50 mg per dose. For creams, gels, ointments, etc., the concentration of the phosphodiesterase inhibitor is generally in the range of about 0.05 wt. % to about 5 wt. %.

It is a further object of the invention to provide a method for treating sexual dysfunction in a female individual by applying a pharmaceutical composition as described herein to the vulvar region and/or vagina of the individual, so as to deliver a therapeutically effective amount of a phosphodiesterase inhibitor.

One or more additional active agents, which may or may not be vasoactive agents, are administered along with the phosphodiesterase inhibitor. The active agents may be simultaneously administered, either in the same composition or dosage form or in different compositions or dosage forms, or the different active agents may be administered sequentially. Preferably, the additional active agent or agents are given as a pre-medication, or pre-treatment, topically, orally, or using some other route of administration.

The method and compositions of the invention not only enhance female sexual desire and responsiveness in individuals who may be in need of such therapy, but are also useful in improving the tissue health of the female genitalia and preventing vaginal atrophy, preventing pain during intercourse as a result of dyspareunia, and alleviating vaginal itching and dryness associated with dyspareunia and other conditions.

In another aspect of the invention, a packaged kit is provided that includes the pharmaceutical composition of the invention, a container housing the composition during storage and prior to administration, and instructions, e.g., written instructions on a package insert or label, for carrying out drug administration in a therapeutically effective manner. The composition may be packaged in the form of any topically administrable dosage form, e.g., as a cream, gel, ointment or vaginal suppository containing the phosphodiesterase inhibitor. The dosage form may be a unit dosage form, i.e., a dosage form that contains a unit dosage of the active agent, wherein the unit dosage is a therapeutically effective dosage for the treatment of female sexual dysfunction.

Additional objects, advantages and novel features of the invention will be set forth in part in the description that follows, and in part will become apparent to those skilled in the art upon examination of the following, or may be learned by practice of the invention.

### DETAILED DESCRIPTION OF THE INVENTION

Definitions:

Before describing the present invention in detail, it is to be understood that this invention is not limited to delivery of specific drugs, carriers or use of particular drug delivery systems, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a phosphodiesterase inhibitor” includes not only a single phosphodiesterase inhibitor but also a combination, e.g., a mixture, of two or more phosphodiesterase inhibitors, reference to “a pharmaceutically acceptable carrier” includes a single such carrier as well as a combination, e.g., a mixture, of two or more pharmaceutically acceptable carriers, and the like.

In describing and claiming the present invention, the following terminology will be used in accordance with the definitions set out below. In describing and claiming the present invention, the following terminology will be used in accordance with the definitions set out below.

The terms “active agent” and “pharmacologically active agent” are used interchangeably herein to refer to a chemical compound that induces a desired pharmacological, physiological effect, in this case, treatment of female sexual dysfunction. The primary active agents herein are phosphodiesterase inhibitors. The terms “active agent” and “pharmacologically active agent” also encompass pharmaceutically acceptable, pharmacologically active derivatives of those active agents specifically mentioned herein, including, but not limited to, salts, esters, amides, inclusion complexes, prodrugs, metabolites, analogs, and the like. When the terms “active agent,” “pharmacologically active agent,” “phosphodiesterase inhibitor,” and any other term referring to a specific drug or drug type are used, then, it is to be understood that the active agent per se is intended as well as pharmaceutically acceptable, pharmacologically active salts, esters, amides, inclusion complexes, prodrugs, metabolites, analogs, etc.

“Carriers” or “vehicles” as used herein refer to carrier materials suitable for vulvar and/or vaginal drug administration, and include any such materials known in the art, e.g., any liquid, gel, solvent, liquid diluent, solubilizer, or the like, which is nontoxic and does not interact with other components present in the pharmaceutical composition or drug delivery system in a deleterious manner.

By an “effective” amount or a “therapeutically effective amount” of an active agent is meant a nontoxic but sufficient amount of the agent to provide the desired effect, i.e., treatment of female sexual desire and responsiveness. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” sexual dysfunction, as the term is used herein, encompasses both prevention of sexual dysfunction in clinically asymptomatic individuals and treatment of dysfunction in a clinically symptomatic individual. It will be appreciated that “treatment of sexual dysfunction” as the term is used herein includes the treatment of disorders of female sexual desire and/or response, meaning any disorder or dysfunction that causes a decrease in or absence of female sexual responsiveness or female sexual desire. This includes any persistent or recurrent deficiency in the desire for sexual activity. It also includes decreases in the physiological response to sexual stimulation such as slowed or decreased erectile response of the female erectile tissues; slowed, decreased or absent lubrication of the vagina; slowed, decreased, or absent ability to have orgasms; decreased intensity of or pleasure in orgasms: frigidity; sexual aversion; and disorders of female sexual desire and response that are secondary to a general medical condition such as the menopausal or post-menopausal state, radiotherapy of the pelvis, atherosclerosis, pelvic trauma or surgery, peripheral neuropathies, autonomic neuropathies, diabetes mellitus, and disorders of the innervation of any of the sexual organs. Treatment of female sexual dysfunction also includes treatment of substance-induced sexual dysfunction, including but not limited to, decreases in desire and responsiveness secondary to anti-depressants, neuroleptics, anti-hypertensives, tobacco, opiates, alcohol and any other drug found to decrease or eliminate any part of the sexual response cycle. Primary and secondary anorgasmia are included.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well, i.e., therapeutically effective for the treatment of premature ejaculation.

By “as-needed” dosing, also referred to as “pro re nata” dosing, “prn” dosing, and “on-demand” dosing or administration, is meant the administration of an active agent at some time prior to anticipated sexual activity and within a time interval sufficient to provide for the desired therapeutic effect, i.e., enhancement in sexual desire and in sexual responsiveness during sexual activity. Preferably, “as-needed” administration herein does not involve priming doses or chronic administration. As-needed administration will generally be in the range of about 1 minute to about 60 minutes prior to anticipated sexual activity, more typically in the range of about 5 minutes to about 30 minutes prior to anticipated sexual activity. “As-needed” administration may also involve administration of a sustained release formulation in advance of anticipated sexual activity, with drug release taking place throughout an extended drug delivery period.

The term “controlled release” is intended to refer to any drug-containing composition in which release of the drug is not immediate. The term is used interchangeably with “nonimmediate release” as defined in Remington: The Science and Practice of Pharmacy, Nineteenth Ed. (Easton, Pa.: Mack Publishing Company, 1995). Thus, the term “controlled release” is used in the conventional sense to include any nonimmediate release formulation, including, but not limited to, sustained release, delayed release and pulsatile release formulations.

The term “sustained release” is used in its conventional sense to refer to a drug formulation that provides for gradual release of a drug over an extended period of time.

The term “topical administration” is used in its conventional sense to mean delivery of a topical drug or pharmacologically active agent to the skin or mucosa. Topical administration thus includes transmucosal administration, and transmucosal administration, in the context of the present invention, includes both vulvar administration and vaginal delivery. Vaginal delivery involves direct administration of a pharmaceutical formulation to the vagina of the individual undergoing treatment, and generally involves administration to the distal several centimeters of the vagina. The terms “vulvar delivery” and “vulvar administration” are used herein to refer to application of a pharmaceutical composition to the vulvar area of an individual undergoing treatment. The terms are intended to encompass application to the clitoris as well as the surrounding vulvar area. The terms “vulvar delivery” and “clitoral delivery” are used interchangeably herein and are both intended to refer to administration to the vulvar area of the individual undergoing treatment.

The primary active agent herein is a phosphodiesterase inhibitor. Suitable phosphodiesterase inhibitors include, but are not limited to, inhibitors of the type III phosphodiesterases (cAMP-specific-cGMP inhibitable form), the type IV phosphodiesterases (high affinity-high specificity cAMP form) and the type V phosphodiesterases (the cGMP specific form).

Suitable Type III inhibitors include, but are not limited to, those described in U.S. Pat. No. 6,156,753 to Doherty, Jr. et al., assigned to VIVUS, Inc. (Mountain View, Calif.). Such inhibitors include, by way of example: bipyridines such as milrinone, amrinone and olprinone; imidazolones such as piroximone and enoximone; imidazolines such as imazodan and 5-methyl-imazodan; midazoquinoxalines; dihydropyridazinones such as indolidan and LY181512 (5-(6-oxo-1,4,5,6-tetrahydro-pyridazin-3-yl)-1,3-dihydro-indol-2-one); dihydroquinolinone compounds such as cilostamide, cilostazol, vesnarinone, and OPC 3911 (N-cyclohexyl-N-hydroxymethyl-4-(2-oxo-1,2-dihydroquinolin-6-yloxy)-butyramide); other compounds such as anagrelide, bemoradan, ibudilast, isomazole, lixazinone, motapizone, olprinone, phthalazinol, pimobendan, quazinone, siguazodan and trequinsin; and mixed Type III and Type IV inhibitors such as benafentrine, cis-6-[p-acetamidophenyl]-1,2,3,4,4a,10b-hexahydro-8,9-dimethoxy-2-methylbenzo-[c] [1,6]-naphthyridine, EMD 54622 (5-[1-(3,4-dimethoxybenzoyl)-4,4-dimethyl-1,2,3,4-tetrahydrochinolin-6-yl]-6-methyl-3,6-dihydro-1,3,4-thiadiazin-2-one), Org 20241 (N-hydroxy-4-[3,4-dimethoxyphenyl]-thiazole-2-carboximidamide), Org30029 (N-hydroxy-5,6-dimethoxybenzo-[b]-thiophene-2-carboximidamide), saterinone, tolafentrine and zardaverine.

Suitable Type IV inhibitors include, but are not limited to, those described in U.S. Pat. No. 6,127,363 to Doherty, Jr. et al., also assigned to VIVUS, Inc. Examples of Type IV inhibitors that can be administered in conjunction with the present method include, by way of example: pyrrolidinones such as rolipram (4-(3-cyclopentyloxy-4′-methoxyphenyl)-2-pyrrolidinone)) and rolipram derivatives such as RO20-1724 (4-(3-butyloxy-4-methoxyphenyl)-imidazolidinone) and RS 33793 (8-(3-nitrophenyl)-6-(3-methyl-2-butenyl)pyrido-[2,3a]pyrazin-5-one); quinazolinediones such as nitraquazone (3-[3′-nitrophenyl] N-ethylquinazoline-2,6-dione), CP-77059 (1-(carbomethoxyphenyl)-3-benzyl-pyrido[2,3d] pyrimidine-2,4(1H,3H)dione), RS-25344 (1-(3-nitrophenyl)-3-(4-pyridylmethyl)-1,2,3,4-tetrahydro pyrido(2,3-d) pyrimidine-2,4-dione)) and other nitraquazone analogs; xanthine derivatives such as denbufylline (1,3-di-n-butyl-7-[2′-oxopropyl] xanthine), XT-44 (1-n-butyl-3-n-propylxanthine), arofylline (LAS 31025; 1-propyl-3-(4-chlorophenyl)-xanthine) and BRL 61063; phenyl ethyl pyridines such as CDP 840 (4-(1-(3-cyclopentyloxy-4-methoxyphenyl)-2-phenylethyl) pyridine) and compounds disclosed in WO 97/22585 to Guay et al.; tetrahydropyrimidones such as atizoram (CP 80633); diazepine derivatives such as CI 1018 and compounds disclosed in WO 97/36905 to Pascal et al.; oxime carbamates such as filaminast (PDA-641); naphthyridinones such as RS 17597; benzofurans such as 2-butyl-7-methoxy-benzofuran-4-carboxylic acid (3,5-dichloropyridin-4yl)-amide, 2-benzyl-7-methoxy-benzofuran-4-carboxylic acid (3,5-dichloropyridin-4-yl)-amide, 7-methoxy-2-phenethyl-benzofuran-4-carboxylic acid (3,5-dichloropyridin-4-yl)-amide and 5-(2-butyl-7-methoxy-benzofuran-4-yl)-tetrahydro-pyrimidin-2-one, phenanthridines, such as those disclosed in U.S. Pat. No. 6,191,138 to Gutterer; 2-heteroaryl and 2-heterocyclic benzoxazoles, such as those disclosed in U.S. Pat. No. 6,166,041 to Cavalla et al.; phenyldihydro-benzofurane compounds such as those disclosed in U.S. Pat. No. 5,902,824 to Ulrich; benzofuran carboxamides as disclosed in U.S. Pat. No. 6,211,203 to Amschler et al.; 4-substituted benzofurane compounds such as those disclosed in EP 819688A1; substituted furans as disclosed in Perrier et al. (1999) Bioorg. Med. Chem. Lett. 9:323-326 (1999); naphthalene derivatives such as T 440; purine derivatives such as V 112294A and those compounds disclosed in U.S. Pat. No. 6,228,859 to Cavalla et al.; cyclohexane carboxylic acids such as ariflo (SB 207499, c-4-cyano-4-[3′-cyclopentyloxy-4′-methoxyphenyl]-r-1-cyclohexane-carboxylic acid); benzamides such as piclamilast (RP73401; N-(3,5-dichloro-4-pyridyl)-3-cyclopentoxy-4-methoxybenzamide); benzothiophenes such as tibenelast (LY 186655); pyridopyridazinones such as 8-(3-nitrophenyl)-6-pyridin-4-ylmethyl-6H-pyrido[2,3-d]pyridazin-5-one; imidazolidinones such as 5-[3-bicyclo[2.2.1]hept-2-yloxy)-4-methoxyphenyl-1-methyl-imidazolidin-2-one; substituted phenyl compounds, as disclosed in U.S. Pat. No. 5,891,896 to Warrellow et al.; substituted biphenyl compounds as disclosed in U.S. Pat. No. 5,877,190 to Dhainaut et al.; etazolate; and S-(+)-glaucine ((S)-(+)-1,2,9,10-tetramethoxy-aporphine).

Examples of type V phosphodiesterase inhibitors include, but are not limited to, pyrazolopyrimidinones such as those disclosed in PCT Publication No. WO 94/28902, and griseolic acid derivatives, 2-phenylpurinone derivatives, phenylpyridone derivatives, fused and condensed pyrimidines, pyrimidopyrimidine derivatives, purine compounds, quinazoline compounds, phenylpyrimidinone derivative, imidazoquinoxalinone derivatives, and other compounds disclosed in WO 96/16644.

Suitable pyrazolopyrimidinones, as disclosed in WO 94/28902, are 5-(2-ethoxy-5-morpholinoacetylphenyl)-1-methyl-3-n-propyl-1,6-dihydro-7H-pyrazolo[4,3-d]pyrimidin-7-one, 5-(5-morpholinoacetyl-2-n-propoxyphenyl)-1-methyl-3-n-propyl-1,6-dihydro-7H-pyrazolo[4,3-d]pyrimidin-7-one, 5-[2-ethoxy-5-(4-methyl-1-piperazinylsulfonyl)-phenyl]-1-methyl-3-n-propyl-1,6-dihydro-7H-[4,3-d]pyrimidin-7-one, 5-[2-allyloxy-5-(4-methyl-1-piperazinylsulfonyl)-phenyl]-1-methyl-3-n-propyl-1,6-dihydro-7H-pyrazolo[4,3-d]pyrimidin-7-one, 5-[2-ethoxy-5-[4-(2-propyl)-1-piperazinylsulfonyl)-phenyl]-1-methyl-3-n-propyl-1,6-dihydro-7H-pyrazolo [4,3-d]pyrimidin-7-one, 5-[2-ethoxy-5-[4-(2-hydroxyethyl)-1-piperazinylsulfonyl)phenyl]-1-methyl-3-n-propyl-1,6-dihydro-7H-[4,3-d]pyrimidin-7-one, 5-[5-[4-(2-hydroxyethyl)-1-piperazinylsulfonyl]-2-n-propoxyphenyl]-1-methy 1,3-n-propyl-1,6-dihydro-7H-pyrazolo[4,3-d]pyrimidin-7-one, 5-[2-ethoxy-5-(4-methyl-1-piperazinylcarbonyl)phenyl]-1-methyl-3-n-propyl-1,6-dihydro-7H-pyrazolo[4,3-d]pyrimidin-7-one and 5-[2-ethoxy-5-(1-methyl-2-imidazolyl)phenyl]-1-methyl-3-n-propyl-1,6-dihydro-7H-pyrazolo[4,3-d]pyrimidin-7-one.

The griseolic acid derivatives, 2-phenylpurinones, etc. disclosed in WO 96/16644 include 1,3-dimethyl-5-benzylpyrazolo[4,3-d]pyrimidine-7-one, 2-(2-propoxyphenyl)-6-purinone, 6-(2-propoxyphenyl)-1,2-dihydro-2-oxypyridine-3-carboxamide, 2-(2-propoxyphenyl)-pyrido[2,3-d]pyrimid-4(3H)-one, 7-methylthio-4-oxo-2-(2-propoxyphenyl)-3,4-dihydro-pyrimido[4,5-d]pyrimidine, 6-hydroxy-2-(2-propoxyphenyl)pyrimidine-4-carboxamide, 1-ethyl-3-methylimidazo[1,5a]quinoxalin-4(5H)-one, 4-phenylmethylamino-6-chloro-2-(1-imidazoloyl)quinazoline, 5-ethyl-8-[3-(N-cyclohexyl-N-methylcarbamoyl)-propyloxy]-4,5-dihydro-4-oxo-pyrido[3,2-e]-pyrrolo[1,2-a]pyrazine, 5′-methyl-3′-(phenylmethyl)-spiro[cyclopentane-1,7′(8′H)-(3′H)-imidazo[2,1-b]purin]4′(5 ′H)-one, 1-[6-chloro-4-(3,4-methylenedioxybenzyl)-aminoquinazolin-2-yl)piperidine-4-carboxylic acid, (6R,9S)-2-(4-trifluoromethylphenyl)methyl-5-methyl-3,4,5,6a,7,8,9,9a-octa hydrocyclopent[4,5]-imidazo[2,1-b]-purin-4-one, 1-t-butyl-3-phenylmethyl-6-(4-pyridyl)pyrazolo[3,4-d]-pyrimid-4-one, 1-cyclopentyl-3-methyl-6-(4-pyridyl)-4,5-dihydro-1H-pyrazolo[3,4-d]pyrimid-4-one, 2-butyl-1-(2-chlorobenzyl)6-ethoxy-carbonylbenzimidaole, 2-(4-carboxy-piperidino)-4-(3,4-methylenedioxy-benzyl)amino-6-nitroquinazoline and 2-phenyl-8-ethoxycycloheptimidazole.

Still other type V phosphodiesterase inhibitors useful in conjunction with the method of the present invention include: those compounds described in PCT Publication No. WO 01/19802 to Aoyama (Tanabe Seiyaku Co., Ltd.), particularly (S)-2-(2-hydroxymethyl-1-pyrrolidinyl)-4-(3-chloro-4-methoxybenzylamino)-5-[N-(2-pyrimidinylmethyl)carbamoyl]pyrimidine, 2-(5,6,7,8-tetrahydro-1,7-naphthyridin-7-yl)-4-(3-chloro-4-methoxybenzylamino)-5-[N-(2-morpholinoethyl)carbamoyl]-pyrimidine, and (S)-2-(2-hydroxymethyl-1-pyrrolidinyl)-4-(3-chloro-4-methoxy-benzylamino)-5-[N-(1,3,5-trimethyl-4-pyrazolyl)carbamoyl]pyrimidine; zaprinast (1,4-dihydro-5-(2-propoxyphenyl)-7H-1,2,3-triazolo[4,5-d]pyrimidin-7-one); 1-(3-chloroanilino)-4-phenylphthalazine (MY5445); dipyridamole, vinpocetine; FR229934 (Fujisawa Pharmaceutical Co., Ltd.); 1-methyl-3-isobutyl-8-(methylamino)xanthine; tadalafil (IC-351; Cialis®); vardenafil (Bayer); 4-aryl-1-isoquinolinone derivatives such as methyl 2-(4-aminophenyl)-1,2-dihydro-1-oxo-7-(2-pyridinylmethoxy)-4-(3,4,5-trimethoxyphenyl)-3-isoquinoline carboxylate dihydrochloride and the sulfate salt thereof (T-1032); 4-bromo-5-(pyridylmethylamino)-6-[3-(4-chlorophenyl)propoxy]-3 (2H)pyridazinone; 1-[4-[(1,3-benzodioxol-5-ylmethyl)amino]-6-chloro-2-quinazolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]imidazo[2,1-b]purin-4-one; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5-(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidine carboxylic acid, monosodium salt; Pharmaprojects No. 4516 (Glaxo Wellcome), having the molecular structure

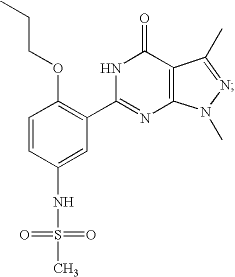

Pharmaprojects No. 5064 (Kyowa Hakko; see WO 96/26940), having the molecular structure

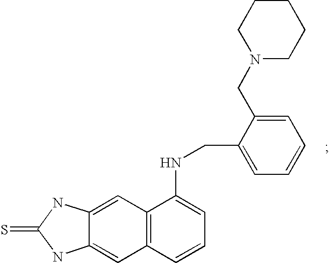

Pharmaprojects No. 5069 (Schering Plough), having the molecular structure

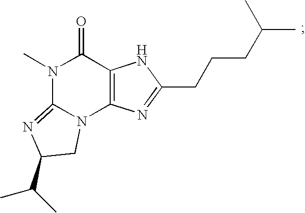

Sch-51866, having the molecular structure

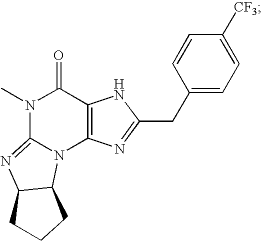

GF-196960 (Glaxo Wellcome); sodium 1-[6-chloro-4-(3,4-methylenedioxybenzyl)-aminoquinazolin-2-yl]piperidine-4-carboxylate sesquihydrate (E4021); and 6-phenylpyrazolo[3,4-d]pyrimidinones such as 1,3-dimethyl-6(2-propoxy-5-methanesulfonamidophenyl)-1,5-dihydropyrazolo[3,4-d]pyrimidin-4-one and 1-ethyl-3-methyl-6-(2-propoxy-5-(4-methylthiazol-2-yl)phenyl-1,5-dihydropyrazolo[3,4-d]pyrimidin-4-one.

Additional type V phosphodiesterase inhibitors useful in conjunction with the method of the present invention include: those compounds described in PCT Publication No. WO 01/19802 to Aoyama (Tanabe Seiyaku Co., Ltd.), particularly (S)-2-(2-hydroxymethyl-1-pyrrolidinyl)-4-(3-chloro-4-methoxybenzylamino)-5-[N-(2-pyrimidinylmethyl)carbamoyl]pyrimidine, 2-(5,6,7,8-tetrahydro-1,7-naphthyridin-7-yl)-4-(3-chloro-4-methoxybenzylamino)-5-[N-(2-morpholinoethyl)carbamoyl]-pyrimidine, and (S)-2-(2-hydroxymethyl-1-pyrrolidinyl)-4-(3-chloro-4-methoxy-benzylamino)-5-[N-(1,3,5-trimethyl-4-pyrazolyl)carbamoyl]pyrimidine.

Particularly preferred Type V phosphodiesterase inhibitors for use in conjunction with the present invention are sildenafil (5-[2-ethoxy-5-(4-methyl-1-piperazinylsulphonyl)phenyl]-1-methyl-3-n-propyl-1,6-dihydro-7H-pyrazolo[4,3-dipyrimidin-7-one]), tadalafil, vardenafil, zaprinast, dipyridamole, and the compounds of WO 01/19802 to Aoyama, particularly those identified above.

Other phosphodiesterase inhibitors include nonspecific inhibitors such as theophylline, theobromine, IBMX, pentoxifylline and papaverine.

One or more additional active agents can be administered with the phosphodiesterase inhibitor, either simultaneously or sequentially. The additional active agent will generally although not necessarily be one that is effective in treating female sexual dysfunction, and/or an agent that potentiates the effect of the phosphodiesterase inhibitor.

Additional pharmacologically active agents may be co-administered along with the primary active agent, i.e., with the phosphodiesterase inhibitors. Such additional active agents, also referred to herein as “secondary” active agents, are generally selected from prostaglandins, angiotensin-converting enzyme (ACE) inhibitors, angiotensin II receptor antagonists, α-receptor blocking agents (“α-blockers”), β2-adrenergic agonists, other adrenoreceptor agents, nitrovasodilators, endothelin-derived relaxation factors, muscarinic agents, dopaminergic agonists, opioid antagonists, ergot alkaloids, polypeptide neurotransmitters, agents that stimulate adenylate cyclase, smooth muscle relaxants, calcium channel blockers, leukotriene inhibitors, phosphodiesterase (PDE) inhibitors, steroids, and steroid antagonists and partial

Suitable prostaglandins may be naturally occurring prostaglandins, semisynthetic prostaglandins, or synthetic prostaglandins. Preferred prostaglandins include, but are not limited to, the naturally occurring prostaglandins PGE₀ (13,14-dihydro-PGE₁), PGE₁, 19-hydroxy-PGE₁, PGE₂, 19-hydroxy-PGE₂, PGA₁, 19-hydroxy-PGA₁, PGA₂, 19-hydroxy-PGA₂, PGB₁, 19-hydroxy-PGB₁, PGB₂, 19-hydroxy-PGB₂, PGB₃, PGD₂, PGF_{1α}, PGF_{2α}(dinoprost), PGE₃, PGF₃Q, PGI₂ (prostacyclin), and combinations thereof. PGE₀, PGE₁, PGE₂, and the hydrolyzable lower alkyl esters thereof (e.g., the methyl, ethyl and isopropyl esters) are, however, particularly preferred. Other prostaglandins useful in conjunction with the method and compositions of the invention are exemplified, without limitation, by arboprostil, carbaprostacyclin, carboprost tromethamine, dinoprost tromethamine, dinoprostone, enprostil, iloprost, lipoprost, gemeprost, metenoprost, sulprostone, tiaprost, viprostil (CL 115,347), viprostil methyl ester, 16,16-dimethyl-Δ²-PGE, methyl ester, 15-deoxy-16-hydroxy-16-methyl-PGE, methyl ester (misoprostol), 16,16-dimethyl-PGE₁, 11-deoxy-15-methyl-PGE₁, 16-methyl-18,18,19,19-tetrahydrocarbacyclin, 16(RS)-15-deoxy-16-hydroxy-16-methyl-PGE, methyl ester, (+)-4,5-didehydro-16-phenoxy-α-tetranor-PGE₂ methyl ester, 1-deoxy-11α,16,16-trimethyl-PGE₂, (+)-11α,16α,16∃-dihydroxy-1,9-dioxo-1-(hydroxymethyl)-16-methyl-trans-prostene, 9-chloro-16,16-dimethyl-PGE₂, 16,16-dimethyl-PGE₂, 15(S)-15-methyl-PGE₂, 9-deoxy-9-methylene-16,16-dimethyl-PGE₂, potassium salt, 19(R)-hydroxy-PGE₂, and 11-deoxy-16,16-dimethyl-PGE₂.

Angiotensin-converting enzyme (“ACE”) inhibitors include, for example, captopril, enalapril, enalaprilat, quinapril, lisinopril, and ramipril. ACE inhibitors may enhance the efficacy of the present formulation and method and decrease long-term complications, such as inflammatory and fibrotic responses. α-Blockers include, but not limited to, prazosin, phentolamine, phenoxybenzamine, dibenamine, doxazosin, terazosin, trimazosin, tolazoline, corynthanine, rauwolscine, piperoxan, alfuzosin, tamsulosin, indoramin, and are especially desirable for increasing the efficacy and prolonging the action of the present method. Other adrenoreceptor agents, including but not limited to, yohimbine, labetalol, carvedilol, and bucindolol, may also enhance the activity and prolong the action of the present method.

Nitrovasodilators include, but are not limited to, nitroglycerin, isosorbide dinitrate, isosorbide mononitrate, amyl nitrate, molsidomine, linsidomine chlorhydrate (“SIN-1”), S-nitroso-N-acetyl-d,l-penicillamine (“SNAP”), S-nitroso-N-glutathione (“SNO-GLU”), erythrityl tetranitrate, sodium nitoprusside, and the diazenium diolates, or “NONOates.” NONOates include, but are not limited to, (Z)-1-{N-methyl-N-[6-(N-methyl-ammoniohexyl)amino]} diazen-1-ium-1,2-diolate (“MAHMA/NO”), (Z)1[N-(3-ammoniopropyl)-N-(n-propyl)amino]diazen-1-ium-1,2-diolate (“PAPA/NO”), (Z)1-{N-[3-aminopropyl]-N-[4-(3-aminopropylammonio)butyl]amino} diazen-1-ium-1,2-diolate (spermine NONOate or “SPER/NO”) and sodium (Z)-1-(N,N-diethylamino)-diazen-1-ium-1,2-diolate (diethylamine NONOate or “DEA/NO”) and derivatives thereof).

Examples of suitable muscarinic agents are pilocarpine, edrophonium, and bethanacol, and representative dopaminergic agonists are apomorphine and bromocriptine. Opioid antagonists include naloxone, naltrexone, nalmefene, nalorphine, methyl naltrexone, CTOP diprenorphine, P-funaltrexamine, naloxonazine, norbinaltorphimine. natrindole, BNTX, and other analogs that exhibit opioid antagonistic properties.

Exemplary ergot alkaloids include ergotamine and ergotamine analogs, e.g., acetergamine, brazergoline, bromerguride, cianergoline, clanegollone, delorgotrile, disulergine, ergonovine maleate, ergotamine tartrate, etisulergine, lergotrile, lysergide, mesulergine, metergoline, metergotamine, nicergoline, pergolide, propisergide, proterguride, and terguride.

Preferred polypeptide neurotransmitters are vasoactive polypeptide neurotransmitters and include, for example, vasoactive intestinal polypeptide (VIP), VIP agonists (VIP analogs), derivatives of VIP and VIP agonists, particularly hydrolyzable lower alkyl esters, as well as calcitonin, calcitonin gene-related products, cholecystokinin and cholecystokinin analogs (e.g., CCK8). Other suitable secondary agents include: forskolin and water-soluble analogues thereof, which directly stimulate adenylate cyclase; dibutyryl-cyclic AMP; dibutyryl-cyclic GMP; and guanylin.

The secondary agent is not necessarily, however, a vasoactive agent. For example, the formulations may contain a steroid or a steroid agonist, partial agonist, or antagonist, in addition to the phosphodiesterase inhibitor.

Steroids will generally be selected from the group consisting of progestins, estrogens, androgens, and combinations thereof. Suitable progestins include, but are not limited to, acetoxy-pregnenolone, allylestrenol, anagestone acetate, chlormadinone acetate, cyproterone, cyproterone acetate, desogestrel, dihydrogesterone, dimethisterone, ethisterone (17α-ethynyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, noretbisterone acetate, norethynodrel, norgestimate, norgestrel, norgestrienone, normethisterone, and progesterone. Progesterone, cyproterone acetate, norethindrone, norethindrone acetate and levonorgestrel are preferred progestins. Suitable estrogens include synthetic and natural estrogens such as: estradiol (i.e., 1,3,5-estratriene-3,17β-diol, or “β-estradiol”) and its esters, including estradiol benzoate, valerate, cypionate, heptanoate, decanoate, acetate and diacetate; 17α-estradiol; ethynylestradiol (i.e., 17α-ethynylestradiol) and esters and ethers thereof, including ethynylestradiol 3-acetate and ethynylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. Estradiol and ethynylestradiol are particularly preferred. Suitable androgenic agents include, but are not limited to: the naturally occurring androgens and derivatives thereof, including androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, dromostanolone propionate, testosterone, dehydroepiandrosterone (DHEA; also termed prasterone), sodium dehydroepiandrosterone sulfate, 4-dihydrotestosterone (DHT; also termed stanolone), and 5α-dihydrotestosterone; pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters; and pharmaceutically acceptable derivatives of testosterone such as methyl testosterone, testolactone, oxymetholone and fluoxymesterone. Testosterone and testosterone esters, such as testosterone enanthate, testosterone propionate and testosterone cypionate, are particularly preferred.

Examples of preferred steroid antagonists or partial agonists are tamoxifen, cenchroman, clomiphene, droloxifene, raloxifene, and pharmaceutically acceptable salts thereof, particularly tamoxifen or clomiphene citrate.

Particularly desirable secondary active agents are E-series prostaglandins (particularly PGE₀, PGE₁, PGE₂, and hydrolyzable lower alkyl esters thereof) and phosphodiesterase inhibitors, α-blockers and/or androgenic steroids.

Any of the active agents may be administered in the form of a salt, ester, amide, inclusion complex, prodrug, metabolite, analog, or other derivative, and such derivatives may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from an active agent in the form of a free base (typically wherein the neutral form of the drug has a neutral —NH₂ group) using conventional means, involving reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on a drug (e.g., the carboxylic acid functionality of prostanoic acid) are prepared in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO-moiety at the carbonyl carbon. Esterification may also be carried out by reaction of a hydroxyl group with an esterification reagent such as an acid chloride. Esters can be reconverted to the free acids, if desired, by using conventional hydrolysis procedures. Amides may be prepared from esters, using suitable amine reactants, or they may be prepared from an anhydride or an acid chloride by reaction with ammonia or a lower alkyl amine.

Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system. Inclusion complexes are complexes formed by interaction of macrocyclic compounds containing an intramolecular cavity of molecular dimensions with the smaller, pharmacologically active agent. Preferred inclusion complexes are formed from α-, β- and γ-cyclodextrins, or from clathrates, in which the “host” molecules form a crystal lattice containing spaces in which “guest” molecules (e.g., the active agent) will fit. See, e.g., Hagan, Clathrate Inclusion Compounds (New York: Reinhold, 1962). Prodrugs, active metabolites and other derivatives can be prepared using techniques known to those of ordinary skill in the art and/or using syntheses described in the pertinent texts and literature.

The primary active agent herein, i.e., the phosphodiesterase inhibitor, may be incorporated into and administered in any composition or dosage form suitable for administration to the vulvar region and/or vagina. If a secondary active agent is also administered topically, it may be incorporated into the same type of composition or dosage form, either along with the phosphodiesterase inhibitor, or in a separate composition or dosage form. These compositions and dosage forms will typically contain one or more pharmaceutically acceptable carriers suited to the particular type of formulation, i.e., gel, ointment, suppository, or the like. The carriers are comprised of materials of naturally occurring or synthetic origin that do not adversely affect the active agent or other components of the formulation. Suitable carriers for use herein include water, silicone, waxes, petroleum jelly, polyethylene glycol, propylene glycol, liposomes, sugars such as mannitol and lactose, and a variety of other materials, again depending, on the specific type of formulation used. Pharmaceutical compositions and dosage forms used for administration to the vulvar region and/or vagina may be used to deliver drug on an as-needed, on-demand basis, and/or throughout an extended, sustained release profile.

The compositions used herein may be in the form of an ointment, cream, emulsion, lotion, gel, solid, solution, suspension, foam or liposomal composition; such formulations may be used for clitoral, vulvar or vaginal delivery. Alternatively, the compositions may be contained within a vaginal ring, tampon, suppository, sponge, pillow, puff, or osmotic pump system; these platforms are useful solely for vaginal delivery.

As is well known in the art, ointments are semisolid preparations that are typically based on petrolatum or other petroleum derivatives. The specific ointment base to be used, as will be appreciated by those skilled in the art, is one that will provide for optimum drug delivery. As with other carriers or vehicles, an ointment base should be inert, stable, nonirritating and nonsensitizing. As explained in Remington: The Science and Practice of Pharmacy, supra, at pages 1034-1038, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy for further information.

Lotions are preparations that may be applied without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. It is generally necessary that the insoluble matter in a lotion be finely divided. Lotions will typically contain suspending agents to produce better dispersions as well as compounds useful for localizing the active agent in contact with the skin, e.g., methylcellulose, sodium carboxymethylcellulose, or the like.

Pharmaceutical emulsion formulations are generally formed from a dispersed phase (e.g., a pharmacologically active agent), a dispersion medium and an emulsifying agent. If desired, emulsion stabilizers can be included in the formulation as well. A number of pharmaceutically useful emulsions are known in the art, including oil-in-water (o/w) formulations, water-in-oil (w/o) formulations and multiple emulsions such as w/o/w or o/w/o formulations. Emulsifying agents suitable for use in such formulations include, but are not limited to, TWEEN 60®, Span 80®, cetostearyl alcohol, myristyl alcohol, glyceryl monostearate and sodium lauryl sulfate.

Pharmaceutical creams, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant.

The above pharmaceutical formulations are formed by dispersing the finely divided or dissolved active agent uniformly throughout the vehicle or base using conventional techniques, typically by a levigating the agent with a small quantity of the base to form a concentrate, which is then diluted geometrically with further base. Alternatively, a mechanical mixer may be used. Creams, lotions and emulsions are formed by way of a two-phase heat system, wherein oil-phase ingredients are combined under heat to provide a liquified, uniform system. The aqueous-phase ingredients are separately combined using heat. The oil and aqueous phases are then added together with constant agitation and allowed to cool. At this point, concentrated agents may be added as a slurry. Volatile or aromatic materials can be added after the emulsion has sufficiently cooled. Preparation of such pharmaceutical compositions is within the general skill of the art.

The agent can also be incorporated into a gel formulation using known techniques. Two-phase gel systems generally comprise a suspension or network of small, discrete particles interpenetrated by a liquid to provide a dispersed phase and a liquid phase. Single-phase gel systems are formed by distributing organic macromolecules uniformly throughout a liquid such that there are no apparent boundaries between the dispersed and liquid phases. Suitable gelling agents for use herein include synthetic macromolecules (e.g., Carbomers, polyvinyl alcohols and polyoxyethylene-polyoxypropylene copolymers), gums such as tragacanth, as well as sodium alginate, gelatin, methylcellulose, sodium carboxymethylcellulose, methylhydroxyethyl cellulose and hydroxyethyl cellulose. In order to prepare a uniform gel, dispersing agents such as alcohol or glycerin can be added, or the gelling agent can be dispersed by trituration, mechanical mixing or stirring, or combinations thereof.

Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N[1-2,3-dioleyloxy)propyl]-N,N,N-triethylammonium (“DOTMA”) liposomes are available under the tradename Lipofectin® (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (“DOPC”), dioleoylphosphatidyl glycerol (“DOPG”), and dioleoylphoshatidyl ethanolamine (“DOPE”), among others. These materials can also be mixed with DOTMA in appropriate ratios. Methods for making liposomes using these materials are well known in the art.

Vaginal suppositories are typically manufactured with polyethylene glycol (PEG), polyethylene oxide and/or other low melting point or water-soluble polymers including fatty acid esters. Suppositories may also be applied to the vulvar region, in which case these dosage forms, which are solid at ambient temperature, rapidly melts when placed on the clitoris and within the vulvar region.

Typically, compositions and dosage forms for vulvar and/or vaginal administration will contain the phosphodiesterase inhibitor at a concentration such that an effective unit dosage of the active agent is delivered with a single application of the composition. An effective unit dosage will depend on the phosphodiesterase inhibitor, the dosage form, and other factors, but will generally be in the range of about 5 μg to about 125 mg per dose, more typically in the range of about 50 μg to about 50 mg per dose. In the present compositions, the concentration of the phosphodiesterase inhibitor is generally in the range of about 0.05 wt. % to about 5 wt. %. Since drug dosages typically vary from person to person, however, repeated applications may be used to achieve the desired effect.

Delivery of an “as-needed” or “on-demand” dose with topical formulations intended for application to the vulvar region, and/or with vaginal suppositories, is effected by using the appropriate carrier and, when necessary, excipients, for the particular active agent. Additionally, one or more permeation enhancers and/or detergents may also be present in the formulation to ensure a rate of delivery sufficient for on-demand administration. A combination of these approaches as well as other approaches may be used to effect delivery of an on-demand dose. Again, by an “as-needed” or “on-demand” dose is meant a dose 1 to 60 minutes prior to anticipated sexual activity, more typically 5 to 30 minutes prior to anticipated sexual activity.

The present compositions and dosage forms may also be designed to provide sustained release of the active agent or agents, e.g., pulsatile, continuous or cyclical drug release. Such systems include, for example, osmotic release systems. More commonly, however, sustained release compositions and dosage forms herein are manufactured with a gradually bioerodible (typically hydrolyzable) polymer. Sustained release of the active agent may follow an initial release “pulse,” in which case release of the active agent can be controlled by bioerosion of a polymer using either encapsulated dissolution control or matrix dissolution control. In encapsulated dissolution control, the dose of the active agent is located within an outer polymeric or wax membrane that dissolves to provide an initial release of the agent. When the encapsulating membrane comprising an initial release of the agent has dissolved, a core containing additional active agent is then available for release and adsorption across the epithelial or mucosal surfaces of the vagina or vulvar area. Bioerodible coating materials may be selected from a variety of natural and synthetic polymers, depending on the agent to be coated and the desired release characteristics. Exemplary coating materials include gelatins, carnauba wax, shellacs, ethylcellulose, cellulose acetate phthalate and cellulose acetate butyrate. Following initial release of the agent, a uniform sustained release of the agent can be attained by compressing a population of particles of the agent with varying membrane thickness (e.g., varying erosion times) into a tablet form for a single administration.

In matrix dissolution control, the active agent is dissolved or dispersed within a matrix of, for example, an erodible wax. The agent is released for adsorption across the epithelial or mucosal surfaces of the vagina or vulvar area as the matrix bioerodes. The rate of agent availability is generally controlled by the rate of penetration of the dissolution media (i.e., vaginal fluids) into the matrix, wherein the rate of penetration is dependent on the porosity of the matrix material. Bioerodible matrix dissolution delivery systems can be prepared by compressing the active agent with a slowly soluble polymer carrier into a tablet or suppository form. There are several methods of preparing drug/wax particles including congealing and aqueous dispersion techniques. In congealing methods, the active agent is combined with a wax material and either spray-congealed, or congealed and then screened. For an aqueous dispersion, the active agent/wax combination is sprayed or placed in water and the resulting particles collected. Matrix dosage formulations can be formed by compaction or compression of a mixture of active agent, polymer and excipients. Of course, the active agent will also be located in an external coating of the matrix formulation to provide for immediate release of the active agent necessary for on-demand administration.

In an alternative embodiment, the pharmaceutical formulation is administered in the form of biodegradable adhesive film or sheet that adheres to the vulvar area. Such drug delivery systems are generally composed of a biodegradable adhesive polymer based on a polyurethane, a poly(lactic acid), a poly(glycolic acid), a poly(ortho ester), a polyanhydride, a polyphosphazene, or a mixture or copolymer thereof. Preferred biodegradable adhesive polymers include polyurethanes and block copolyurethanes containing peptide linkages, simple mixtures of polyurethanes and polylactides, and copolymers of acrylates and mono- or disaccharide residues.

The present compositions and dosage forms may contain one or more pharmaceutically acceptable excipients, i.e., additives that are not pharmacologically active but rather impart some desirable characteristic to the formulations. Suitable excipients are known to those skilled in the art and include, for example, detergents, permeation enhancers, antioxidants, pH-adjusting agents, and buffers.

A suitable detergent is one that increases the solubility of the active agent in the composition or dosage form and therefore the bioavailability of the agent following administration. The detergent will typically be a nonionic, anionic, cationic or amphoteric surfactant. In the practice of the invention, the surfactant is selected such that local irritation at the site of administration is avoided. Examples of suitable surfactants include Tergitol® and Triton® surfactants (Union Carbide Chemicals and Plastics, Danbury, Conn.), polyoxyethylene sorbitans, e.g., TWEEN® surfactants (Atlas Chemical Industries, Wilmington, Del.), and pharmaceutically acceptable fatty acid esters such as lauryl sulfate and the like.

Permeation enhancers are chemical compounds that enhance permeation of the active agent through the body surface, and are useful herein in conjunction with the administration of those active agents that have a relatively low rate of permeation through mucosal membranes. Suitable permeation enhancers include those generally useful in conjunction with topical, transdermal or transmucosal drug delivery. Examples of suitable permeation enhancers include dimethylsulfoxide (DMSO), dimethyl formamide (DMF), N,N-dimethylacetamide (DMA), decylmethylsulfoxide (C₁₀MSO), polyethylene glycol monolaurate (PEGML), glycerol monolaurate, lecithin, the 1-substituted azacycloheptan-2-ones, particularly 1-n-dodecylcyclazacycloheptan-2-one (available under the trademark Azone® from Nelson Research & Development Co., Irvine, Calif.), lower alkanols (e.g., ethanol), SEPA® (available from Macrochem Co., Lexington, Mass.), and surfactants as discussed above, including, for example, Tergitol®, Nonoxynol-9® and TWEEN-80®.

The formulation may also include a compound to reduce oxidation of one or more of the components in the formulation. Antioxidants are well known to those skilled in the art. Suitable antioxidants for use in the formulations described herein include butylhydroxytoluene (BHT), ascorbic acid and its sodium and potassium salts, the citrate salts such as sodium, potassium, or ammonium citrate, and tocopherol. The amount of the antioxidant will vary according to the particular antioxidant, amount of the formulation, and other variables. A suitable antioxidizing amount of an antioxidant for any particular formulation can be determined by one of ordinary skill in the art.

The compositions and dosage forms may also include one or more pH-adjusting agents. A sufficient amount of a pharmaceutically acceptable acid or base, e.g., HCl or NaOH, may be added to aqueous formulations described herein in order to adjust the pH to the desired value. For nonaqueous formulations, adjusting the pH may be accomplished by adding unit doses of a residual powder obtained by lyophilization of a buffered aqueous solution, e.g., an aqueous solution of a salt of an organic acid, the aqueous solution having the desired pH. Such a buffered aqueous solution may be advantageously prepared with a citrate salt such as sodium citrate. Upon contact with a mucosal membrane, the lyophilized salt solution will buffer the pH of the mucosal fluid to a pH within the desired range, thereby enhancing the activity of the PGE₂ as the PEG melts. Additionally, the proper amount of the pH-adjusting agent can be determined experimentally by adding incremental amounts of the pH-adjusting agent and serially testing the formulation using a conventional pH meter until the formulation has the desired pH.

Preferred dosage forms contain a unit dose of active agent, i.e., a single therapeutically effective dose. For creams, ointments, etc., a “unit dose” requires an active agent concentration that provides a unit dose in a specified quantity of the formulation to be applied, as explained supra. The unit dose of any particular active agent will depend, of course, on the active agent, the needs of the patient, and on the mode of administration, although, as noted previously, the preferred dose of the phosphodiesterase inhibitor is in the range of about 1 μg to about 5,000 μg, more typically about 20 μg to about 2,000 μg. Those of ordinary skill in the art of pharmaceutical formulation can readily deduce suitable unit doses for various secondary agents from the pertinent texts and literature. Drug administration may be on an as-needed basis (about 1 to 60 minutes prior to anticipated sexual activity, more typically 5 to 30 minutes prior to anticipated sexual activity). With a sustained release dosage form, a single “as-needed” dose can provide therapeutic efficacy over an extended time period in the range of about 4 to 72 hours, typically in the range of about 4 to 48 hours, more typically in the range of about 4 to 24 hours, depending on the formulation. The release period may be varied by the selection and relative quantity of particular sustained release polymers. It is not necessary, in either case, to carry out chronic drug administration, i.e., regular dosing on an ongoing basis (such as on a weekly basis, twice weekly, daily, etc.).

As-needed administration as described herein has several advantages over chronic pharmacologic intervention. First, chronic administration of some agents, in particular steroids, can result in serious medical complications and alter the balance of naturally occurring steroids in the body. Second, patient compliance can be problematic with a regimented dosing scheme. Furthermore, as-needed administration is convenient and doses are taken only in anticipation of sexual activity. Thus, needless expenditure on wasted dosages is avoided, thereby decreasing the treatment's overall expense.

The pharmaceutical compositions and dosage forms may be administered vaginally and/or by application to the vulvar region. Often, the formulation will be administered to the clitoris, which is often retracted or hidden under the clitoral hood. Thus, prior to administration, the clitoral hood may be retracted with the finger of one hand, and the clitoral hood should be held back as the dose is applied. Additionally, the formulation may be applied to the tissues covering the clitoris, such as the prepuce and the frenulum of the labiaminora, with massaging in order to achieve application to the clitoris.

The patient treated is typically a woman suffering from some type of sexual dysfunction or disorder, or may possess “normal” sexual desire and/or “normal” sexual responsiveness as those terms are understood defined by clinicians or other experts. For these “normal” women, the present invention offers heightened sexual desire and responsiveness relative to her typical sexual experience, and substantially prevents sexual dysfunction. Usually, however, the female patient seeking enhanced sexual desire and responsiveness suffers from a sexual dysfunction such as a condition, disease or disorder that affects one of the four stages of the female sexual response: excitement, plateau, orgasm, or resolution. More specifically, the patient may suffer from any one or more of the following:

a decrease in or absence of female sexual responsiveness or female sexual desire;

a persistent or recurrent deficiency or absence of sexual fantasies and desire for sexual activity;

a decrease in the physiological response to sexual stimulation such as, but not limited to, slowed, decreased or absent erectile response of the female erectile tissues; slowed, decrease or absent lubrication of the vagina; slowed decreased or absent ability to reach orgasm, and decreased intensity of or pleasure in orgasms;

frigidity;

sexual aversion;

a condition, disease or disorder that may result in decreased sexual desire and responsiveness including, but not limited to, the menopausal or post-menopausal state, radiotherapy of the pelvis, multiple sclerosis, atherosclerosis, pelvic trauma or surgery, peripheral neuropathies, autonomic neuropathies, diabetes mellitus, and disorders of the innervation of any of the sexual organs;

substance-induced decreases in sexual desire and responsiveness including, but not limited to, decreases related to the administration of pharmacologic agents such as, but not limited to, anti-depressants, neuroleptics, anti-hypertensives, opiates, alcohol, and any other agent found to decrease or eliminate any part of the sexual response cycle; and

primary and secondary anorgasmia.

In addition to enhancing female sexual desire and responsiveness, the method, compositions and dosage forms of the invention are useful in improving the tissue health of the female genitalia and preventing vaginal atrophy, preventing pain during intercourse as a result of dyspareunia, and alleviating vaginal itching and dryness associated with dyspareunia and other conditions.

In another embodiment, a packaged kit is provided that contains: the composition or dosage form to be administered, i.e., a pharmaceutical formulation containing a phosphodiesterase inhibitor for treating female sexual dysfunction; a container (e.g., a vial, a bottle, a pouch, an envelope, a can, a tube, an atomizer, an aerosol can, etc.), preferably sealed, for housing the formulation during storage and prior to use; and instructions for carrying out drug administration in a manner effective for the treatment of female sexual dysfunction. The instructions will typically be written instructions on a package insert, a label, and/or on other components of the kit.

Depending on the type of formulation and the intended mode of administration, the kit may also include a device for administering the formulation. The administration device may be a dropper, a swab, a stick, or the nozzle or outlet of an atomizer or aerosol can. The formulation may be any suitable composition or dosage form as described herein. For example, the composition may be in the form of a vaginal suppository, in the form of a gel, cream or ointment contained within a tube, or in the form of a solution or lotion in a vial. The kit may contain multiple formulations of different dosages of the same agent, and/or multiple formulations of different active agents.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, patent publications and non-patent literature references mentioned herein are incorporated by reference in their entireties.

### EXPERIMENTAL

The practice of the invention will employ, unless otherwise indicated, conventional techniques of pharmaceutical formulation and the like, which are within the skill of the art. Such techniques are fully explained in the literature. See, for example, Remington. The Science and Practice of Pharmacy, supra. In particular, the formulations should be prepared under aseptic conditions. In addition, many formulations, e.g., suppository formulations, are preferably stored in a range from about 3° C. to about 8° C., and have a melting point preferably in the range from about 15° C. to about 38° C., more preferably from about 21° C. to about 32° C. Furthermore, repeated thawing of frozen formulations should be avoided as the formulation may deteriorate or the active agent may become inactivated. Optimally, the formulations will be protected from light.

In the following examples, efforts have been made to ensure accuracy with respect to numbers used (e.g., amounts, temperature, etc.) but some experimental error and deviation should be accounted for. Unless indicated otherwise, temperature is in degrees C. and pressure is at or near atmospheric pressure at sea level. All reagents were obtained commercially unless otherwise indicated.

### Example 1

A cream formulation is prepared for the topical administration of sildenafil. The cream includes the following components:

| | | |
|---|---|---|
| Sildenafil | 500 | mg |
| Beeswax | 2.7 | g |
| Carbopol ® 934 q.s. | 100.0 | g |

Mixing is conducted with tile and spatula until a homogeneous cream mixture is obtained having the active agent uniformly dispersed throughout the composition.

### Example 2

The procedure of Example 1 is repeated except that the following components are used:

| | | |
|---|---|---|
| Sildenafil | 3.5 | g |
| Polyethylene glycol 400 | 37.5 | g |
| 1,2,6-hexanetriol | 20.0 | g |
| Polyethylene glycol 4000 q.s. | 100.0 | g |

A homogenous cream mixture is obtained.

### Example 3

The procedure of Example 1 is repeated except that the following components are used:

| | | |
|---|---|---|
| Tadalafil | 1.0 | g |
| Polyethylene glycol 400 | 37.0 | g |
| Polyethylene glycol 400 monostearate | 26.0 | g |
| Polyethylene glycol 4000 q.s. | 100.0 | g |

A homogenous cream mixture is obtained.

### Example 4

The procedure of Example 1 is repeated except that the following components are used:

| | | |
|---|---|---|
| TA-1790 | 2.5 | g |
| Polyethylene glycol 400 | 47.5 | g |
| Cetyl Alcohol | 5.0 | g |
| Polyethylene glycol 4000 q.s. | 100.0 | g |

A homogenous cream mixture is obtained.

### Example 5

A vaginal suppository formulation is prepared for the administration of sildenafil. The suppository includes the following components:

| | | |
|---|---|---|
| Sildenafil | 5.0 | g |
| Polyethylene glycol 400 | 37.0 | g |
| Glycerol gelatin | 20.0 | g |
| Polyethylene glycol 4000 q.s. | 100.0 | g |

The polyethylene glycol 400 solution containing the active agent is mixed well with glycerol gelatin with a mechanical mixing apparatus and heated to form a liquid. The higher molecular weight polyethylene glycol is added, and the mixture is then cooled in a suppository mold.

### Example 6

A vaginal suppository formulation A gel preparation of sildenafil was prepared by admixing 2.5 g sildenafil with 100 g of high purity soybean lecithin (Sigma Chemical Company, St. Louis, Mo.). Then, water was slowly added with agitation until a thick, viscous gel was produced.

### Example 7

A liposomal solution is prepared as follows. Either an aqueous or oil-based solution of tadalafil and an optional additional active agent can be added to a liposomal mixture of, for example, 10 g of safflower oil, 10 g of soybean oil, 1.2 g of egg phosphatides, and 2.5 g of glycerin in a final volume of 100 ml (the remainder being water). Two mg of tadalafil may be added, and the resulting mixture then stirred until all components are dissolved. Liposomal solutions are particularly favored for active agents with limited solubility in water.

### Example 8

Individuals are assessed and pre-screened to assemble an experimental group of subjects suffering from sexual dysfunction. The compositions prepared in Examples 1-7, formulated with various phosphodiesterase inhibitors, are each assessed in the experimental subjects for their ability to increase uterine or vaginal epithelial blood flow. The formulations are applied vaginally, and changes in blood flow or vaginal fluid production after application of the vasodilating formulations are determined using known methods. Increase in vaginal epithelial blood flow is determined using indirect methods such as photoplethysmography (Levin (1980) Clinics in Obstet. Gynaecol. 7:213-252), heated oxygen electrode (Wagner et al. (1978), “Vaginal Fluid” in The Human Vagina, Evans et al. (eds.), Amsterdam: Elsevier/North Holland Biomedical Press, pp. 121-137), and direct clearance of radioactive Xenon (Wagner et al. (1980) Obstet. Gynaecol. 56:621-624). Changes in vulvar blood flow are monitored using laser Doppler velocimetry (Sarrel, P. M. (1990) Obstet. Gynaecol. 75:26S-32S).

Decreased vaginal dryness and/or dyspareunia are negatively correlated with vaginal blood flow rates, wherein increased blood flow to the vagina correlates with increased lubrication and decreased frequency and severity of dyspareunia (Sarrel, P. M. (1990) Obstet. Gynaecol. 75:26S-32S). Accordingly, vulvar blood flow after treatment is assessed using laser Doppler velocimetry and compared to baseline levels. Increased vaginal lubrication as a result of treatment with the topical phosphodiesterase inhibitor formulations can also be assessed using the methods of Semmens et al. (1982) J. Am. Med. Assoc. 248:445-448. The formulations of Examples 1-7, when assessed using such methods, are found to substantially increase blood flow to the vagina and vulvar area and alleviate vaginal dryness.

We claim:

1. A pharmaceutical formulation for treating sexual dysfunction in a female individual, comprising a therapeutically effective amount of a phosphodiesterase inhibitor and a pharmaceutically acceptable carrier suited to vaginal and/or vulvar drug administration.

2. The pharmaceutical formulation of claim 1, wherein the phosphodiesterase inhibitor is selected from the group consisting of Type III phosphodiesterase inhibitors, Type IV phosphodiesterase inhibitors, Type V phosphodiesterase inhibitors, nonspecific phosphodiesterase inhibitors, and combinations thereof.

3. The pharmaceutical formulation of claim 2, wherein the phosphodiesterase inhibitor is a Type III phosphodiesterase inhibitor.

4. The pharmaceutical formulation of claim 3, wherein the Type III phosphodiesterase inhibitor is selected from the group consisting of bipyridines, imidazolones, imidazolines, dihydropyridazinones, quinolinones, anagrelide, bemoradan, isomazole, lixazinone, pimobendan, siguazodan and trequinsin.

5. The pharmaceutical formulation of claim 3, wherein the Type III phosphodiesterase inhibitor is selected from (5-(4-acetamidophenyl)pyrazin-2-(1H)-one), amrinone, anergrelide, bemoradan, cilostamide, cilostazol, enoximone, imazodan, indolidan, isomazole, lixazinone, 5-methylimazodan, milrinone, pimobendan, piroximone, siguazodan, trequensin, and vesnarinone.

6. The pharmaceutical formulation of claim 2, wherein the phosphodiesterase inhibitor is a Type IV phosphodiesterase inhibitor.

7. The pharmaceutical formulation of claim 6, wherein the Type IV phosphodiesterase inhibitor is selected from the group consisting of rolipram, 4-(3-butyloxy-4-methoxyphenyl)-imidazolidinone (RO20-1724), nitraquazone, 1-(carbomethoxyphenyl)-3-benzylpyrido[2,3d] pyrimidine-2,4(1H,3H)dione (CP-77059), 1-(3-nitrophenyl)-3-(4-pyridylmethyl)-1,2,3,4-tetrahydro pyrido(2,3-d) pyrimidine-2,4-dione (RS-25344-00), denbufylline, etazolate, 5-[1-(3,4-dimethoxybenzoyl)-4,4-dimethyl-1,2,3,4-tetrahydrochinolin-6-yl]-6-methyl-3,6-dihydro-1,3,4-thiadiazin-2-one (EMD54622), arofylline, and derivatives thereof.

8. The pharmaceutical formulation of claim 2, wherein the phosphodiesterase inhibitor is a Type V phosphodiesterase inhibitor.

9. The pharmaceutical formulation of claim 8, wherein the Type V phosphodiesterase inhibitor is selected from the group consisting of pyrazolopyrimidinones, griseolic acid derivatives, 2-phenylpurinones, phenylpyridone derivatives, pyrimidines, pyrimidopyrimidines, purines, quinazolines, quinazolinones, quinoxalines, benzimidazoles, cycloheptimidazoles, phenylpyrimidinones, imidazoquinazolines, and imidazoquinoxalinones or aza analogues thereof.

10. The pharmaceutical formulation of claim 8, wherein the Type V phosphodiesterase inhibitor is a pyrazolopyrimidinone.

11. The pharmaceutical formulation of claim 10, wherein the Type V phosphodiesterase inhibitor is selected from sildenafil and acid addition salts thereof.

12. The pharmaceutical formulation of claim 8, wherein the Type V phosphodiesterase inhibitor is tadalafil.

13. The pharmaceutical formulation of claim 8, wherein the Type V phosphodiesterase inhibitor is zaprinast.

14. The pharmaceutical formulation of claim 8, wherein the Type V phosphodiesterase inhibitor is dipyridamole.

15. The pharmaceutical formulation of claim 8, wherein the Type V phosphodiesterase inhibitor is vardenafil.

16. The pharmaceutical formulation of claim 8, wherein the Type V phosphodiesterase inhibitor is selected from (S)-2-(2-hydroxymethyl-1-pyrrolidinyl)-4-(3-chloro-4-methoxy-benzylamino)-5-[N-(2-pyrimidinylmethyl)carbamoyl]pyrimidine, 2-(5,6,7,8-tetrahydro-1,7-naphthyridin-7-yl)-4-(3-chloro-4-methoxybenzylamino)-5-[N-(2-morpholinoethyl)carbamoyl]-pyrimidine, and (S)-2-(2-hydroxymethyl-1-pyrrolidinyl)-4-(3-chloro-4-methoxy-benzylamino)-5-[N-(1,3,5-trimethyl-4-pyrazolyl)carbamoyl]-pyrimidine.

17. The pharmaceutical formulation of claim 2, wherein the phosphodiesterase inhibitor is a nonspecific phosphodiesterase inhibitor.

18. The pharmaceutical formulation of claim 17, wherein the phosphodiesterase inhibitor is selected from the group consisting of theophylline, IBMX, pentoxifylline and papaverine.

19. The pharmaceutical formulation of claim 1, contained within a delivery system that provides a predetermined agent release profile.

20. The pharmaceutical formulation of claim 19, wherein the delivery system provides for sustained release of the phosphodiesterase inhibitor.

21. The pharmaceutical formulation of claim 1, wherein the therapeutically effective amount of the phosphodiesterase inhibitor is effective to treat female sexual dysfunction when the formulation is administered vaginally, and further wherein the carrier is suited to vaginal drug administration.

22. The pharmaceutical formulation of claim 21, in the form of an ointment, cream, gel, solid, solution, suspension, foam or liposomal composition.

23. The pharmaceutical formulation of claim 22, wherein the pharmaceutical formulation is contained within a vaginal ring, tampon, suppository, sponge, pillow, puff, or osmotic pump system.

24. The pharmaceutical formulation of claim 1, wherein the therapeutically effective amount of the phosphodiesterase inhibitor is effective to treat female sexual dysfunction when the formulation is administered to the vulvar area, and further wherein the vehicle is suited to vulvar drug administration.

25. The pharmaceutical formulation of claim 1, further including an additional active agent selected from the group consisting of additional vasodilators, androgenic agents, steroid antagonists, steroid agonists and partial agonists, and combinations thereof.

26. The formulation of claim 25, wherein the additional active agent is an additional vasodilator.

27. The formulation of claim 26, wherein the additional vasodilator is selected from the group consisting of prostaglandins, endothelial-derived relaxation factors, vasoactive intestinal polypeptide agonists, smooth muscle relaxants, leukotriene inhibitors, and combinations thereof.

28. The formulation of claim 27, wherein the additional vasodilator is a prostaglandin.

29. The formulation of claim 28, wherein the prostaglandin is PGE₀, PGE₁, PGE₂, or a combination thereof.

30. The formulation of claim 29, wherein the prostaglandin is PGE₁.

31. The pharmaceutical formulation of claim 25, wherein the additional active agent is an androgenic agent.

32. The formulation of claim 31, wherein the androgenic agent is selected from the group consisting of testosterone, dehydroepiandrosterone, Sa-dihydrotestosterone, methyl testosterone, testolactone, fluoxymesterone, and pharmaceutically acceptable salts and esters thereof.

33. The formulation of claim 32, wherein the androgenic agent is selected from the group consisting of testosterone, 5α-dihydrotestosterone, and pharmaceutically acceptable esters thereof.

34. The formulation of claim 33, wherein the androgenic agent is testosterone.

35. The formulation of claim 34, wherein the androgenic agent is Sa-dihydrotestosterone.

36. The formulation of claim 33, wherein the androgenic agent is a testosterone ester.

37. The formulation of claim 36, wherein the testosterone ester is selected from the group consisting of testosterone enanthate, testosterone propionate, testosterone cypionate, testosterone phenylacetate, testosterone acetate, testosterone isobutyrate, testosterone buciclate, testosterone heptanoate, testosterone decanoate, testosterone undecanoate, testosterone caprate and testosterone isocaprate.

38. The formulation of claim 33, wherein the androgenic agent is a 5α-dihydrotestosterone ester.

39. The formulation of claim 38, wherein the 5α-dihydrotestosterone ester is selected from the group consisting of the 5α-dihydrotestosterone enanthate, 5α-dihydrotestosterone propionate, 5α-dihydrotestosterone cypionate, 5α-dihydrotestosterone phenylacetate, 5α-dihydrotestosterone acetate, 5α-dihydrotestosterone isobutyrate, 5α-dihydrotestosterone buciclate, 5α-dihydrotestosterone heptanoate, 5α-dihydrotestosterone decanoate, 5α-dihydrotestosterone undecanoate, 5α-dihydrotestosterone caprate and 5α-dihydrotestosterone isocaprate.

40. A method for treating sexual dysfunction in a female individual, comprising administering to the vagina and/or vulvar area of the individual a pharmaceutical formulation that comprises an effective amount of a phosphodiesterase inhibitor selected from the group consisting of Type III phosphodiesterase inhibitors, Type IV phosphodiesterase inhibitors, nonspecific phosphodiesterase inhibitors, pharmacologically acceptable salts, esters, analogs, derivatives, prodrugs and inclusion complexes thereof, and combinations of any of the foregoing.

41. A method for improving vaginal muscle tone and tissue health in a female individual, comprising vaginally administering to such individual a pharmaceutical formulation that comprises a phosphodiesterase inhibitor selected from the group consisting of Type III phosphodiesterase inhibitors, Type IV phosphodiesterase inhibitors, nonspecific phosphodiesterase inhibitors, pharmacologically acceptable salts, esters, analogs, derivatives, prodrugs and inclusion complexes thereof, and combinations of any of the foregoing, in combination with a pharmaceutically acceptable carrier.

42. A method for enhancing vaginal lubrication in a female individual, comprising vaginally administering to such individual a pharmaceutical formulation that comprises a phosphodiesterase inhibitor selected from the group consisting of Type III phosphodiesterase inhibitors, Type IV phosphodiesterase inhibitors, nonspecific phosphodiesterase inhibitors, pharmacologically acceptable salts, esters, analogs, derivatives, prodrugs and inclusion complexes thereof, and combinations of any of the foregoing, in combination with a pharmaceutically acceptable carrier.
